(19) United States
(12) Patent Application Publication
(10) Pub. No.: US 2024/0268880 A1
(43) Pub. Date: Aug. 15, 2024

(54) MODULAR ELECTROSURGICAL GENERATOR SYSTEM

(71) Applicant: Olympus Winter & IBE GmbH, Hamburg (DE)

(72) Inventors: Stefan Dietrich, Potsdam (DE); Anne Kwik, Berlin (DE); Daniel Ramin, Nuthetal (DE); Thomas Fähsing, Berlin (DE); Thomas Prezewowsky, Teltow (DE); Stefan Schiddel, Stahnsdorf (DE); Jens Krüger, Zeuthen (DE); Mario Arnold, Berlin (DE); Rocco Arras-Krempien, Königs Wusterhausen/Zernsdorf (DE); Wolfgang Kühne, Schildow (DE); Jelle Dijkstra, Berlin (DE); Fabian Janich, Potsdam (DE); Fabian Stopp, Berlin (DE); Imke Malchow, Neumünster (DE); Veronika Handrick, Berlin (DE)

(73) Assignee: Olympus Winter & IBE GmbH, Hamburg (DE)

(21) Appl. No.: 18/544,653

(22) Filed: Dec. 19, 2023

Related U.S. Application Data
(60) Provisional application No. 63/434,982, filed on Dec. 23, 2022.

(51) Int. Cl. A61B 18/12; A61B 18/00

(52) U.S. Cl. CPC A61B 18/1206; A61B 2018/00178; A61B 2018/1273

(57) ABSTRACT

Modular generator system comprising at least two generator types which are composed of a plurality of generator components and differ with regard to their equipment and/or functions. The generator components comprise a power supply and an inverter for generating an output energy for an electrosurgical instrument. The generator types are each of modular design with a plurality of modules: (i) at least one uniform base module having a communication unit, and (ii) at least one individual module which may differ depending on generator type. Individual modules can be a module for the power supply, a module for the inverter, a module for the output connection. Different generator types differ with regard to their individual module equipment. By virtue of such a two-tier module concept, namely uniform base module and differing individual modules, a maximum number of variants can be achieved with a minimum number of different modules.

The invention relates to a modular generator system comprising at least two generator types of electrosurgical generators. The generator types differ with regard to their equipment and functions and are each designed for feed to at least one electrosurgical instrument.

In electrosurgery or radio-frequency surgery, an electrosurgical instrument, such as an electric scalpel, is used to input energy in the form of high-frequency alternating current into tissue of the human body. In particular frequencies of 200 kHz or more up to 4000 kHz, typically around 400 kHz, are used in this case. In particular, the tissue is cut or severed by way of the heating thus caused. One advantage of this is that, at the same time as the cutting, it is also possible to stem bleeding by closing the affected vessels, and electrosurgical instruments are conceivable for further types of applications, such as for coagulation, for example.

In order to supply a wide variety of indications for patients in line with requirements, electrosurgical generators are needed which are equipped depending on the envisaged field of application and have correspondingly different functions. In this regard, there are type series of different electrosurgical generators for applications in the field, regarding the field in general terms, and for visceral surgery, in more specialized fields such as for ear/nose/throat, urology, gynaecology, gastroenterology, pneumology or phlebology. Within these type series, there are in turn grades for smaller and larger generators, more powerful and less powerful generators, and generators having more or fewer functions. While this is advantageous for the patient and for the surgeon in respect of an electrosurgical generator which is tailored so as to accurately match the respective indication and is of the size desired in each case and has the functional scope desired in each case, this actually means an enormously high number of different variants for the manufacturer.

Providing such a diversity of variants of electrosurgical generators is extremely complex. This great complexity is also an obstacle to modernization or renewal of the generators, since this would have to be carried out separately in each case for the different type series and sizes in a similar, but nonetheless varying form. However advantageous the diversity of variants is for the use of a comprehensive generator system, on the one hand, this may nevertheless be disadvantageous with regard to advancing technology because adaptations and modernizations are made more difficult by the diversity of variants.

The problem addressed by the invention is that of providing an improved generator system which avoids these disadvantages.

The solution according to the invention is found in the features of the independent claims. Advantageous developments are the subject matter of the dependent claims.

In a generator system comprising at least two generator types of electrosurgical generators which differ with regard to their equipment and/or functions, the generator types each being designed for feed to at least one electrosurgical instrument and being composed of a plurality of generator components comprising a power supply and an inverter, fed by the power supply for generating an output energy which is passed to at least one output connection for connection of the electrosurgical instrument, according to the invention it is provided that the generator types are each of modular design with a plurality of modules comprising (i) at least one base module, which is uniform for the different generator types and which comprises at least one communication unit with other modules, and (ii) at least one individual module, which is different depending on generator type, the individual module (s) being embodied at least as module for the power supply and/or module for the inverter and/or at least one module for the output connection, the at least one base module being designed identically for the different generator types, and the different generator types differing with regard to their individual module equipment.

The invention is based on the idea of the generator types each being of modular design and a modular concept thus being created, this modular concept comprising different modules, but also different kinds of modules. In this regard, one kind of module is the base module, which is used uniformly for the different generator types. In this case, “uniformly” means that they are designed substantially identically, in particular with regard to the hardware set-up, where unavoidable or slight adaptations may be provided; differences between ID or encoding devices regarding identity (e.g. serial number) are not ruled out either. —Another kind of module is individual modules, more precisely at least one individual module, where the individual module (s) may differ between the different generator types. By virtue of the module concept being divided into two, namely uniform base module and differing individual modules, it is possible, as recognized by the invention, to achieve a maximum number of variants with a minimum in respect of the number of different modules in order to produce as many different generator types as possible.

In this case, the concept of the base module provides for a common and fundamentally invariable basis to which the individual modules can always relate in the same way. This concerns the required communication of the modules, in particular, for which the base module always provides a communication unit. Consequently, totally independently of the kind and function or size and performance of the individual module, it is ensured that this individual module interacts with a known uniform basis in the form of the base module and can participate in the communication with the base module and, if present, also with other individual modules. In this case, it is unimportant how the respective individual module is constituted or what function it performs. It also does not matter whether the individual module requires electrical energy, as in the case of an inverter, for generating the output energy, or provides this, as in the case of a power supply as individual module. In this way, different generator types can be produced almost universally by way of equipping with different individual modules. Consequently, a high number of variants can be produced and offered with little complexity.

Firstly, an explanation is given of a few terms that are used:

An electrosurgical generator is designed for feeding energy to at least one electrosurgical instrument. This is generally effected by means of high-frequency AC voltage. Typical frequency ranges are in the range of between 200 kHz and 4000 kHz. The high-frequency AC voltage can optionally have amplitudes in the high-voltage range, typically up to 10 kV, preferably up to 4000 V.

The power provided by the electrosurgical generator is typically in the range of between 1 and 500 watts, where the load impedance may vary greatly, and output voltage and energy output may accordingly likewise change greatly and quickly. Consequently, high dynamic power variations occur, but there are also static power variations between different generator types, typically for power requirements beginning with a few watts through to several 100 W.

An inverter used for generating the output energy is understood in the broader sense to mean an inherently arbitrary high-voltage generator. This can be a free-running generator, in particular embodied as a single-ended converter, or else a controlled inverter in the narrower sense, which can generate (high-frequency) output energy with a predefined frequency and (high-voltage) amplitude. The energy generated is typically passed via an output energy line to the connection for the electrosurgical instrument.

Different generator types are considered to be such electrosurgical generators which differ with regard to their equipment and/or function. That can mean, in particular, that the generator types differ with regard to the kind and number of the connections for electrosurgical instruments (in the jargon, these connections are also referred to as “sockets”), and/or they can differ in regard to the available operating modes or cutting modes (which in the jargon are also referred to as “modes”). Furthermore, the generators can differ e.g. in regard to their inverter for generating the output energy; these can differ in particular in terms of the power that they output and also the kind of (high-) voltage generation. That also means that the generator types can be of different sizes, both physically and in regard to their power.

The modules typically each form a physical unit per se. That is understood to mean that they form individually handleable (integral or multipartite) bodies, which is thus easy to handle and to mount. By contrast, the modules typically do not have a dedicated housing, since they do not require that; this is because the modules are to be inserted into the actual housing of the electrosurgical generator and are protected by this. A distinction should be made between that and regions to be shielded which are provided on or at the modules and which can be embodied with a corresponding shield, which can also be of boxlike design, as is typically the case for radio-frequency components. In this regard, it can advantageously be provided that the housing, too, is of modular design and is provided in different sizes (such as small, medium and large), and it can thus be treated similarly to a (specific) individual module.

The generic term “modules” is understood to mean both individual modules and the base module (or, if a plurality of base modules are present, then these as well).

The base module—or, if a plurality thereof are provided, at least one of the base modules—is advantageously embodied as a distribution module having an electricity supply distribution unit in addition to the communication unit. It is designed to distribute the power required for the operation of the modules to the respective connected modules and to communicate accordingly with the other modules via the communication unit. However, the base module need not necessarily be embodied as a distribution module, rather other and/or additional base modules can additionally or alternatively be provided as well. This can involve for example a display control module, an internal distribution module, and/or an external communication module for communication to the outside.

In the case of the individual modules, a plurality of different individual modules are advantageously provided, which differ functionally and thus form kinds of individual modules. Examples of kinds of individual modules are power supplies of the electrosurgical generator, inverters for generating the output energy (high voltage), connections (“sockets”, therefore also referred to as socket modules), in particular connections for electrosurgical instruments, and/or front panel modules, typically comprising operator modules arranged on the front side usually with a display.

Within a kind of individual modules (i.e. for example in the case of the individual module kind “inverters”), different variants can preferably be provided, which differ functionally. The difference can reside in additional functions and/or larger or more powerful design and dimensioning. In this regard, optionally in the case of the individual module kind “inverters”, provision can be made for not just a basic version of an inverter but as well an advanced inverter to be provided, which has control over additional kinds of high-voltage generation, e.g. by means of more complex waveforms (“modes”) or additional selectable waveforms, or has a higher high voltage or, if appropriate, simply just generates more output energy. The same applies, mutatis mutandis, to the individual module kind concerning the power supply; within this kind of modules, provision can be made of variants with lower or higher power, which is output with a fixed or variable voltage, for example, for the purpose of supplying the inverter and optionally also other modules of the electrosurgical generator.

Expediently, provision is made for the individual modules to be individually exchangeable. This constitutes a significant advantage of the modular design with the individual modules, since it is thus easily possible to produce further configurations with different equipment with individual modules. In this way, with little complexity, the manufacturer can produce many different configurations adapted to different requirements and fields of application.

Preferably, at least one optional module with additional functionality is furthermore provided. This module is optionally addable, i.e. can either be added or not. Preferably, it is embodied such that an electricity supply and communication connections to other modules are automatically produced in the mounted state. In this way, optional additional functions can be integrated as modules into the module system according to the invention. One example thereof is, in particular, a module with an ultrasound generator for surgical ultrasound instruments or a module for supplying noble gas, for example an argon module.

Expediently, the modular generator system comprises a plurality of different type series of generator types, for each of which dedicated individual modules are provided, which differ from the individual modules of another type series. In this way, a differentiation between different type series can also be effected by means of the individual modules. This also makes it possible to provide type series graded according to complexity. In this regard, provision can be made of a simple type series having a certain number of individual modules, and a sophisticated type series, in which additional individual modules and also additional kinds of individual modules are provided or can be mounted. In these ways, a finer differentiation of the product range for the generator system according to the invention is made possible, without the complexity increasing unnecessarily. Furthermore, this enables a cost-effective entry-level series to be offered, without the full variability of the module system being dispensed with at the other end. It is particularly advantageous here if the base module is usable across type series. Thus, at a central and crucial location, a standardization is realized and a solid foundation is thus laid which can then be used as a basis for implementing a differentiation across the individual modules.

Referring now once again to the base module aspect, provision is preferably made for the base module or at least one of the base modules to have an electricity supply distribution unit for other modules and/or connectors for the at least one module for the output connection. In this regard, an expedient electricity supply for the modules can be provided, a possibility of accommodation for the output connection module also additionally being created with the connector. Although it will be sufficient, in principle, if just one connector is provided, the base module would then be limited to generator types with just one module for the output connection. By virtue of its having a plurality of connectors, there are thus a plurality of accommodating locations for output connection modules, which also enables variants with a plurality of output connections, specifically by means of the same base module. The connector (s) is/are preferably embodied in each case as plug location or plug locations. Expediently, it is provided that at the connectors has connections for a communication network, an electricity supply network and preferably also the output energy line. This has the effect of achieving a complete supply and linking of the individual modules connected to the connectors, specifically both with regard to the supply with electrical energy and with regard to the integration in the communication network for data communication with other modules and also for connection to electrical signals. It is preferred if the output energy as generated by the inverter as high voltage, in particular, is also applied to the connectors. It goes without saying that such an output energy line is implemented so as to be spatially remote and electrically isolated from the communication network and the electricity supply, in order to avoid an undesirable flashover—which is dangerous for the operational safety of the generator and for the users thereof of the (sometimes high) output voltage required for the output energy and to minimize disturbing interference on account of the high frequency thereof.

Advantageously, it is provided that the modules each have a dedicated communication unit, which in the mounted state with the communication units of the other modules form a communication network for inter-module communication, which is preferably self-configuring. Inter-module communication is understood to mean communication between the modules, i.e. from one module to another. The interface unit ensures that the modules are equipped for, and capable of, independent communication with the other modules. Via the communication network thus formed, the modules can carry out their communication independently for the most part, and so the base module is not necessarily burdened with the provision and implementation of the entire communication with and between the modules. In particular, direct module-to-module communication is made possible, which correspondingly relieves the burden on the base module. It is extremely expedient if the base module likewise participates in the communication network. It can advantageously even be provided that the base module not only participates in the communication network, but even performs a central task of the communication and in this respect can function as master. Advantageously, the communication network is embodied as self-configuring. That means that forming a communication network does not necessarily require the existence of a master which performs the configuration of the individual participants in the communication network or monitors and controls the communication in the communication network.

Expediently, it is provided, at least with respect to the individual modules, that in regard to their communication units and their electricity supply, they have uniform interfaces to the outside to other modules. The individual modules are thus identical outwardly, i.e. towards the other modules, with regard to their interfaces. Exchange for some other individual module, even with a non-identical function, is thus facilitated since this other individual module, despite its different nature, has the same interfaces to the outside, i.e. towards the other modules. However, the communication interfaces can differ with regard to their speed, as explained below.

Advantageously, the communication network is embodied such that it comprises a faster network and a separate slower network. Preferably, the base module and all the individual modules which are critical for the operation of the electrosurgical generator are connected to the fast communication network. Preferably, the faster data network is embodied as a data network with real-time capability. Modules that are connected to the slower network are preferably those which are not critical or are only less critical for the operation of the electrosurgical generator or which in turn require only a small level of data traffic, which is not time-critical. The slower data network is also suitable for connecting subunits to modules or in the case of modules divided into two or more spatially separate units, for their communication among one another. It is expedient if the faster data network is embodied as a CAN network. This enables a high degree of communication with real-time capability. The slower data network can also be embodied as a (slower) CAN network.

Optionally, an extension unit can be provided for the base module. Said extension unit expediently has additional connectors. The necessary prerequisites can thus be created for accommodating additional modules at the additional connectors, in order thus to be able to equip the electrosurgical generator with additional output connection modules, in particular. This is advantageous for generator types with higher-quality equipment levels, in particular, in which a multiplicity of output connections such as monopolar sockets, bipolar sockets, universal sockets and optionally also so-called neutral sockets are provided. Precisely in the case of the sockets, the advantage of the base module with its extension unit is fully manifested since here by way of the base module optionally the (usually high) voltage for the output energy can also be supplied directly, thus obviating additional complexity or wiring that is difficult to insulate in the electrosurgical generator. Preferably, for this purpose, the extension unit also has an output energy line, which—just like at the base module—is designed to carry high voltage for the output energy.

It is possible, as already mentioned, for the base module to be embodied in various ways. An embodiment of the base module as a display control module is based on the idea that this advantageously enables a separation of, firstly, display indicator and, secondly, the display control module. Consequently, the display control module can be embodied as a base module, while a smaller or larger display can be installed as indicator, depending on equipment. Outwardly towards the user, a differentiation is thus achieved, while from a technical standpoint the module used for the control of the display (display control module) remains identical as a base module, which simplifies the set-up and the integration in the communication network and the configuration.

The invention furthermore extends to a modular electrosurgical generator system comprising at least two generator types of electrosurgical generators which differ with regard to their functions, the generator types each being designed for feed to an electrosurgical instrument and being composed of a plurality of generator components comprising a power supply and an inverter, fed by the power supply for generating an output energy which is passed to at least one output connection for connection of the electrosurgical instrument, wherein according to the invention the modular generator system comprises (i) a number of different inverter modules, from which one is selected and mounted for the respective generator type, and (ii) a number of different output connections, at least one of which is selected and mechanically accommodated in a front panel and electrically connected to the mounted inverter, each electrosurgical generator having a module detector, which is designed for recognizing mounted and connected modules and determines therefrom a module configuration really present. In this case, the front panel can likewise be embodied as a module (front panel module), although that is not mandatory.

This aspect of the invention is based on the idea that a number of different modules exist which include at least different inverter modules and different output connections, at least one of each being selected and correspondingly mounted. Moreover, further modules can be provided, such as have been described above in particular as base and/or individual modules. A highly variable set-up or equipping of the electrosurgical generator with modules is made possible in this way. For the operation of the electrosurgical generator, however, it is crucial for there to be clarity about the present equipment with modules (module configuration). For this purpose, a module detector is provided, which is designed for recognizing mounted and connected modules and determines therefrom the module configuration really present. This is expediently done in an automated manner. Thus, during mounting, basically it is possible to select freely from the modules depending on the customer's specific requirements or on the basis of the manufacturer's type series requirements, and the module detector, upon start-up or during operation, detects what modules are actually present and in what configuration they can be used for operation of the electrosurgical generator. Self-checking and self-monitoring are thus afforded.

It goes without saying that module recognition can only take effect in relation to cooperating modules having corresponding means for electronic recognition. Passive components without such electronic means, such as e.g. the housing, cannot and need not be detected.

Furthermore, this can advantageously be utilized for a self-configuration. For this purpose, the modules preferably have a dedicated self-configuration unit designed to configure the respective module depending on the determined module equipment really present. The module equipment really present is understood to mean firstly the modules that are actually physically present, but secondly also the aspect of the extent to which the module is activated in each case. This last is of importance in particular in the case of modules which are the same or similar with regard to their hardware, but for which different firmware is available or the firmware is released to a varying degree in order that different functionalities (generally a small and a larger function set) are thus made possible and made retrievable in a targeted manner. The self-configuration unit determines this and accordingly sets the configuration of its module automatically. What is thus achieved is that the configuration of the modules always corresponds to, or matches, the module equipment actually present and activated. The risk of incorrect configurations and resultant operational disturbances and the risk of damage to the apparatus or harm to the patient are minimized as a result.

The module detector is expediently embodied as a central unit for the respective electrosurgical generator. This has the advantage that information about the module equipment is determined at a central location and is retrievable there. The risk of inconsistencies during the configuration of the individual modules is thus counteracted. The module detector is advantageously arranged on the base module or one of the base modules. This is not mandatory, however.

Provision can also be made, however, for the module detector to be embodied in a decentralized manner, specifically preferably on the respective modules. By this means, the individual modules can each ascertain by themselves in what module environment they are used and the respective configuration of the modules is then based on this information.

Advantageously, a release unit is furthermore provided, which is designed, depending on the module configuration really present, to determine enabled functions and to communicate corresponding release signals to the modules. In this way, the functionality of the individual modules and/or the scope of the functionality can be ascertained and set depending on the module configuration really present. This ensures that each module is configured to match the respective electrosurgical generator and the modules installed there. The risk of incorrect configurations such as were typically able to occur in the case of a conventional manner of configuration is thus effectively prevented.

In this case, the release unit can be embodied as a central unit which centrally performs the corresponding release of the modules. Provision can also be made, however, for the release unit to be embodied in a decentralized manner on the respective modules. Advantages and disadvantages correspond analogously to those as mentioned above concerning the central and decentralized embodiment of the module detector.

Expediently, it is provided that the individual modules are designed to the effect that communication connections and an electricity supply of the modules are automatically produced in the mounted state. In this way, over and above the actual mounting of the modules, there is no need for any independent activity for the modules in regard to production of the communication connections and the electricity supply thereof. This simplifies the use of the modules and also the start-up or initial configuration. The automatic embodiment practically precludes sources of error such as may arise as a result of manual intervention.

The communication connections are usually a data network, additional signal connections preferably being provided for enable signals regarding activity of the modules. By virtue of the modules preferably independently configuring themselves to the communication connections, their participation in the data network and thus the data exchange with the other modules, such as the base module and/or individual modules, is automatically ensured in this way, without further assistance. This simplifies the configuration and reduces the risk of sources of error. The enable signals can furthermore be used to control whether certain modules are active or are activated and thus participate at all. That allows a simple differentiation of the functional equipment by virtue of certain modules or functionalities of the modules being enabled or not being enabled. The module system can thus be refined with regard to its granularity without additional further modules being required for this purpose. Moreover, an additional safety aspect is afforded by virtue of the fact that modules recognized as not belonging or as defective are not released and are thus excluded from the operation of the electrosurgical generator.

Advantageously, the modules each have a dedicated processor unit with a dedicated control program as independent local operational control for the respective module, and they are connected to a generator-internal electricity supply network and communication network. With the dedicated processor and control program, the modules are autonomous in this respect and do not require control externally vis-à-vis the modules, for example by a central unit. By virtue of the modules accomplishing this autonomously by themselves, consequently the data traffic is reduced and the processing speed or speed is increased. Moreover, in this way, depending on module or depending on module complexity, an appropriate processor can be provided so that the performance thereof is always adapted to the requirements of the respective module.

At least the high-voltage generating modules and/or optional ultrasound modules have an additional radio-frequency output connection. With such an additional connection for this particular output energy (high voltage and/or ultrasound), the corresponding modules are also autonomous with regard to the output. The connection paths for this particular output energy are thus shortened and optionally also kept away from the other modules. Furthermore, these modules are also suitable in particular for retrofitting existing electrosurgical generators, specifically those whose output connections are already fully utilized in a different way.

Furthermore, a number of different power supply modules are expediently provided, from which one is selected and mounted for the respective generator type. In this way, in a manner similar to that in the case of the inverter modules already mentioned, a power supply adapted to the size and scope of equipment of the respective electrosurgical generator can be selected and provided in a simple way. Including the power supply in the modular concept results in a considerable increase in the modularization range.

In a particularly expedient embodiment, which merits independent protection if applicable, a protection unit is provided, which is designed to compare a module configuration really present with a unique, permanently stored target equipment feature for the module configuration and, if there is any deviation, to output an error signal, which preferably actuates an operational shutdown. The permanently stored target equipment feature is a data set that is typically defined and permanently stored by the manufacturer itself. This data set represents the configuration of the modules that is provided by the manufacturer. Later, upon start-up or on the part of the customer in the context of routine operation by way of the protection unit of the electrosurgical generator, the module configuration really present (such as is typically ascertained by the module detector) is consulted and compared with the stored target equipment feature. If the module configuration really present corresponds to that in accordance with the target equipment feature, operation is enabled. If there is no correspondence, there is either a defect in the electrosurgical generator or one of its modules or there has been manipulation of the modules or their configuration; then the error signal is output and as a consequence the operation of the electrosurgical generator is advantageously shut down for safety purposes. Optionally, provision can be made for the shutdown to be only partial, such that, if possible, basic operation can still continue to take place.

Advantageously, an upgrade unit is furthermore provided, which is controllable externally via a secure communication connection and interacts with the protection unit and the self-configuration unit of the modules in order to provide the stored target equipment identifier feature with an authorized change and/or to release additional functions. In this way, in an authorized way, it is possible to perform adaptations with regard to the module configuration; furthermore, additional functions can be released. This makes it possible, even after production of the electrosurgical generator, to integrate and utilize additional modules or optionally to extend the functionality of existing modules. The secure communication connection can be a secure remote access. What is thus achieved is that the adaptation of the module configuration can also be effected remotely for example by the manufacturer of the electrosurgical generator. This can take place in the context of an update, for example of the firmware of the modules, or in the context of an upgrade, whereby additional functions are enabled and/or additionally incorporated modules are released.

The invention is explained by way of example below on the basis of an advantageous embodiment with reference to the accompanying drawing, in which:

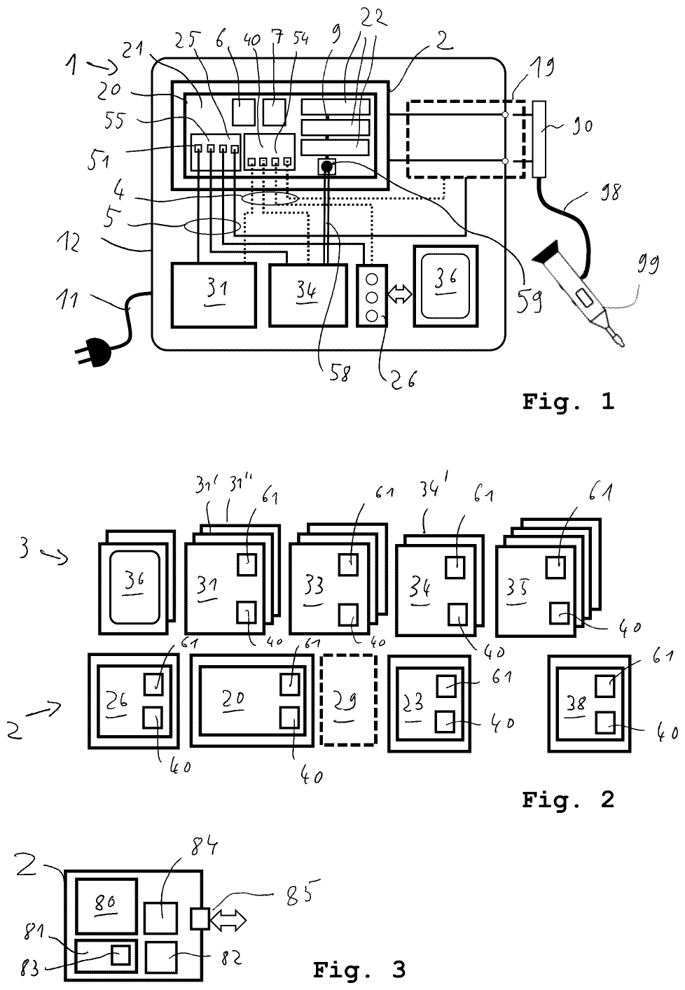

FIG. 1 shows a schematic illustration of an electrosurgical generator in accordance with an exemplary embodiment with a connected electrosurgical instrument;

FIG. 2 shows a block diagram relating to the electrosurgical generator in accordance with FIG. 1;

FIG. 3 shows a schematic illustration for further functional units on the basis of the example of a base module;

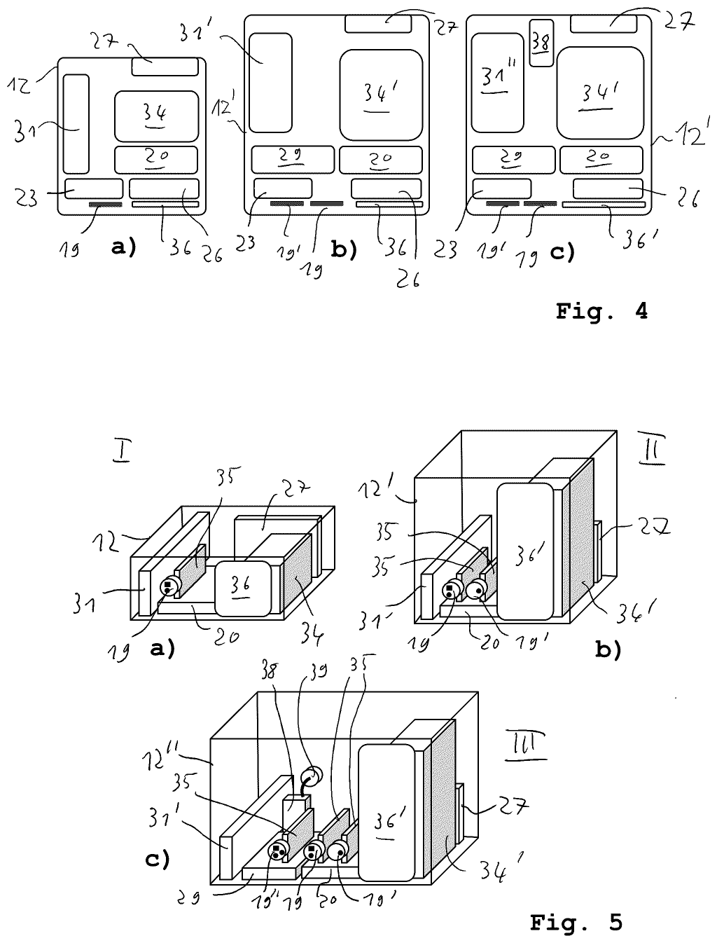

FIGS. 4a-c show schematic illustrations concerning the arrangement of the modules in various housings; and

FIGS. 5a-c show perspective illustrations of various exemplary embodiments concerning the arrangement of the modules in various housings.

An electrosurgical generator in accordance with an exemplary embodiment of the invention is illustrated in FIG. 1. The electrosurgical generator, designated in its entirety by the reference numeral 1, comprises a housing 12, which is provided with an output connection 19 for an electrosurgical instrument 99, which here is an electric scalpel in the exemplary embodiment illustrated. That is connected to an output connection 19 of the electrosurgical generator 1 via a connection plug 90 of a high-voltage connecting cable 98.

The electrosurgical generator 1 is provided with a network connection cable 11, via which it can be connected to the public electricity network and fed therefrom. It should be noted that other sources can also be used for feed purposes, for example a DC voltage feed at 12 V or 48 V in the case of electrosurgical generators 1 which are used in vehicles or in some other environment with mainly DC voltage supply. The network connection cable 11 (or some other kind of feed, such as the DC voltage feed) is attached to a power supply module 31, which provides therefrom the voltages required for operation of the components and modules of the electrosurgical generator 1 and distributes them via an electricity supply network 5 to the individual modules. Thus, by this means, the power supply 31 feeds an inverter 34 for generating the output energy (high voltage) to be output to the electrosurgical instrument 99.

From the energy supplied by the power module 31, the inverter 34 generates a high-frequency AC voltage as output energy. That is typically in the high-voltage range, but can have amplitudes in the range of a few 10 V to 4000 V. The kind of embodiment of the inverter 34 for generating the output energy is open insofar as inherently any of the known concepts, for example single-ended converters or inverters in the narrower sense, can be provided. What is essential is that it generates the output energy required for the electrosurgical instrument 99 in regard to high frequency and likewise high voltage. This output energy generated in this way is passed via the distribution module 20 by means of an output energy line 9 to the output connection 19 for connection of the electrosurgical instrument 99.

For setting the manner of operation and for indicating certain functions, a display control module 26 is provided, which is represented symbolically in the illustration with a user interface having operating buttons, via which the user can set the electrosurgical generator 1 according to the user's desires. It interacts with an indicator embodied as a display module 36.

The components to be provided and their functions are basically known to this extent. The special feature of the invention resides, in particular, in the fact that components of the electrosurgical generator are of modular design. In this respect, reference is made to FIG. 2. The latter illustrates examples of various modules which can be taken as a basis for selecting modules in order to create generator types of electrosurgical generators 1 that differ in terms of equipment and function. The lower row illustrates base modules 2, at least one of which must be present and is typically identical across different electrosurgical generators 1 of a type series.

The base modules 2 include in particular a distribution module 20, which has an electricity supply distribution unit 25 and a communication unit 40 and both distributes the electrical power required for operation to the other modules and correspondingly communicates with the other modules via the communication unit 40 and a communication network 4 connected thereto. It will often be sufficient if provision is made of a single base module 2 in the form of the distribution module 20. However, this is not mandatory; provision can also be made for particularly complex generators 1 preferably from a different type series to be provided with a different base module 2. Further base modules can be in particular the display control module 26 or a rear-side module 27 provided for communication purposes, or a front-side module 23 likewise designed for communication purposes. Optionally, an extension unit 29 can furthermore be provided for the distribution module 20, and is to be coupled only to the distribution module 20, in order thus to provide further connection locations for additional modules of the electrosurgical generator 1. In this way, even generator types having very extensive equipment and many individual modules 3 can be constructed using the same base module 2.

The upper row in FIG. 2 illustrates individual modules which are typically likewise required for operation of the electrosurgical generator 1, but in respect of which there is a choice. In this way, different generator types that differ in regard to equipment and function can be created through the choice of different individual modules 3. The individual modules include, besides the power supply module 31 and the inverter 34, in particular, a front panel module 32, at least one connection module 35 for the connection socket 19, the display module 36.

The base module 2 and the individual modules 3 are each provided with a communication unit 40. Via the latter, the respective module is functionally connected to the communication network 4. The communication network 4 is embodied as a CAN network, specifically divided into two with a faster network and a slower network.

Reference is now made once again to FIG. 1, where a distribution module 20 is illustrated as base module 2. That comprises an electricity supply distribution unit 25 and a communication unit 40 on an optional baseplate 21. Furthermore, connection plugs 54, 55 are provided on the distribution module 20 for the connection between the base module 2 and the individual modules 3.

The power supply module 31 is connected via a supply plug 51. Via the latter, the power provided by the power supply module 31 is passed to the distribution module 20, which then forwards the power to the base and individual modules 2, 3 via the electricity supply distribution unit 25.

In a manner known per se, the inverter 34 generates an AC voltage from the electrical power supplied by the power supply 31, which AC voltage is to be output as output energy to the surgical instrument 99. For this purpose, the AC voltage output by the inverter 34, which is typically a high voltage, is passed via a high-voltage line connection 58 to the distribution module 20, specifically to a high-voltage connection 59 arranged there. From the high-voltage connection 59, the high voltage is applied via an output energy line 9 integrated into the distribution module 20. This voltage is passed inter alia to a plurality of connectors 22 on the distribution module 20, a respective connection module 35 for the electrosurgical instrument 99 being able to be accommodated in the connectors 22 embodied as accommodating locations.

In this way, at least one and optionally a plurality of connection modules 35 can be supplied with the high voltage generated by the inverter 34, namely simply by these connection modules 35 being inserted into their respective connector 22. If e.g. an electrosurgical generator 1 of a different generator type has additional output connections 19′, then for this purpose it is merely necessary to provide additional connection modules 35, which each simply need to be inserted into one of the connectors 22 and are then automatically supplied, specifically also with the high voltage generated by the inverter 34. In this way, even complex electrosurgical generators 1 with a plurality of different connections 19 can be realized modularly in a simple way, namely simply by a corresponding number of connection modules or different connection modules 35 being plugged into one or more of the connectors 22 provided.

It goes without saying that the connectors 22 are likewise connected to the electricity supply distribution unit 25 and communication unit 40 in order to be supplied accordingly (not illustrated in FIG. 1).

Consequently, there is a triple supply, namely for the regular electricity supply, the data communication connection and for the high voltage as output energy, formed in regard to different kinds of individual modules 3 to be connected, including the connection modules 35. The latter are to be inserted directly into the distribution module 20 in the exemplary embodiment described and are then directly connected to the output energy passed as high voltage in the output energy line 9 and are also connected to the electricity supply distribution unit 25 and communication unit 40. The other individual modules 3 are typically connected via cable connections to the electricity supply distribution unit 25 and communication unit 40. For this purpose, they are connected via the communication network 4 to the communication unit 40 and via connection plugs 55 to the electricity supply distribution unit 25 of the distribution module 20. This ensures high flexibility in particular with regard to the spatial arrangement of the individual modules 3, as is advantageous inter alia in regard to housings 12 of different sizes and the hence varying installation position or spacings.

The electrosurgical generator 1 furthermore has a module detector 6. The latter is designed to determine the modules mounted and connected in the electrosurgical generator 1 and the configuration of said modules. The module detector 6 can be arranged in a decentralized manner on the respective modules 2, 3. In the exemplary embodiment illustrated, however, the module detector 6 is arranged centrally, specifically at the distribution module 20 of the base module 2. The module detector 6 is connected via lines (not illustrated in FIG. 1) to the electricity supply network 5 and the communication network 4. The module detector 6 is automated. The module detector 6 communicates with the various modules 2, 3 of the electrosurgical generator 1 via the communication network 4. In this case, the module detector 6 determines which modules 2, 3 are mounted and active. The module detector 6 thus ascertains which modules 2, 3 are actually present and how these modules 2, 3 are configured. In this way, the module detector 6 recognizes the module equipment of the electrosurgical generator 1. This can be utilized for example for recognizing the actual equipment of the electrosurgical generator 1.

The module detector 6 furthermore interacts with a self-configuration unit 61 on the modules 2, 3. For this purpose, the modules 2, 3 each have a self-configuration unit 61. By means of the self-configuration unit 61, the respective module 2, 3 can automatically configure itself, specifically depending on the actual equipment respectively determined, i.e. the module equipment really present in the electrosurgical generator 1. In this regard, for example, it is possible to configure the module 34 for the inverter, which generates the high voltage for the electrosurgical instrument 99, depending on the kind of output connection 19 and the output module 35 thereof. In this regard, the output energy or the kind of outputting of the output voltage, in particular the waveform thereof, that is to say therefore the “modes” of the inverter 34, can be configured automatically depending on the output connection 19 to be supplied and the connection module 35 thereof. The module feeding the inverter 34 for the power supply 31 is then in turn configured by its own self-configuration unit 61 with regard to the voltage or energy to be output, depending on the configuration of the inverter 34 that is set—as described above. In this regard, a practically complete self-configuration of the modules 2, 3 of the electrosurgical generator 1 can be achieved in this way. Courtesy of the self-configuration unit 61 arranged in a decentralized manner, this is possible even if modules 2, 3 are exchanged or a new individual module 3 is added.

Furthermore, a release unit 7 is provided. That is arranged centrally in the exemplary embodiment illustrated, specifically at the distribution module 20. The release unit 7 is designed, depending on the module configuration really present (which is ascertained by the module detector 6, for example), to determine enabled functions and to communicate corresponding release signals to the modules 2, 3. In this way, the functionality of the individual modules 2, 3 overall can be activated or shut down, and/or the scope of their respective functionality can be ascertained and set, specifically depending on the module configuration really present. For this purpose, the release unit 7 communicates corresponding release signals via the communication network 4 to the modules 2, 3. This ensures that only those modules 2, 3 and their functionality which match the respective electrosurgical generator 1 and the configuration with the modules 2, 3 installed there are released and activated.

Reference is now made to FIG. 3, which schematically shows further functional units that can be arranged at the modules 2, 3. By way of example, this is explained on the basis of the base module 2 in FIG. 3; this applies, mutatis mutandis, to the individual modules 3. The base module 2 furthermore comprises a processor unit 80 provided with a dedicated control program. The latter is stored in a memory 81. The base module 2 thus has an independent local operational control, which, like the base module 2, is connected to the generator-internal electricity supply network 5 and communication network 4. This applies, mutatis mutandis, to the individual modules 3 likewise equipped. With the dedicated processor unit 80 and the stored control program, the modules 2, 3 are autonomous in this respect and do not require external control (where the term “external” is relative to the respective module, in the sense of “externally vis-à-vis the respective module”).

Moreover, further functional units can be provided, which can be arranged locally on one (centrally) or more of the modules 2, 3 (in a decentralized manner). In FIG. 3, this is shown by way of example on the basis of the base module 2; the same may apply, mutatis mutandis, to the individual modules 3. Accordingly, a protection unit 82 with regard to the module configuration is furthermore provided. The protection unit 82 is designed to compare a module configuration really present, such as is determined by the module detector 6, for example, with a target equipment feature 83. The target equipment feature 83 is a data set for the configuration of the modules 2, 3 that is provided by the manufacturer. Upon start-up or on the part of the user in the context of routine operation, the protection unit 82 compares the module configuration really present, such as is determined in particular by the module detector 6, with the stored target equipment feature 83. If there is correspondence, the configuration of the electrosurgical generator 1 is okay and operation can take place as envisaged. If a discrepancy arises, however, either a defect in one of the modules 2, 3 or an incorrect configuration of at least one of the modules 2, 3 is present. A corresponding error signal is output, preferably via the data communication network 4, and indicated to the user on the display 36, and as a consequence operation of the electrosurgical generator 1 is shut down for safety purposes. This shutdown can be complete or partial, depending on the discrepancy, and so optionally specific functions are shut down and the electrosurgical generator 1 can continue to be operated with the unaffected functions or its basic functions. All this is done in an automated manner by means of the protection unit 82.

Furthermore, an upgrade unit 84 can be provided, which interacts with a secure communication connection 85. Via the latter, the upgrade unit can communicate with the outside world, and in particular be controlled by an external authorized entity (for example the manufacturer). The upgrade unit interacts with the protection unit 82 and the memory 81 in order to provide the target equipment feature 83 optionally with an authorized change. In this way, additional functions can be released or further additional modules 3 that have been subsequently incorporated can be approved for operation of the electrosurgical generator 1 and be integrated into the latter. FIGS. 4a-c illustrate various generator types with their respective function blocks. The generator type illustrated in FIG. 4a is a basic embodiment. The latter comprises, as base modules 2, a distribution module 20, a display control module 26 and a front-side module 23. Furthermore, a module for the power supply 31, a module for inverter 34, a rear-side module 27 and a display module 36, arranged in the front panel, are provided as individual modules 3. Furthermore, the output connection 19 for the electrosurgical instrument 99 is arranged on the front panel (see FIG. 1). FIG. 4b illustrates a different generator type, which differs from that illustrated in FIG. 4a essentially by virtue of a more powerful power supply module 31′, a larger and more powerful module for the inverter 34′ and a second output connection 19′. For the two output connections 19, 19′, in order to accommodate their respective output modules 35 (not illustrated in FIG. 4), the distribution module 20 is supplemented by an extension unit 29 in order thus to provide at least one additional accommodating location with a connector 22 (not illustrated in FIG. 4). In order to offer enough space for this, a larger housing 12′ is provided. It is evident that in the centre there is a free space remaining, which, if appropriate, courtesy of the modular concept, can optionally be utilized for further equipment with individual modules 3 or an optional module 14 (none of which is illustrated in FIG. 4b). FIG. 4c in turn illustrates a different generator type. The latter differs from that illustrated in FIG. 4b essentially by virtue of the provision of an additional individual module 3 in the form of an ultrasound module 38. In order that this module, too, is sufficiently supplied with power, a different module for the power supply, namely a boosted power supply 31″, is provided.

FIGS. 5a-c illustrate three different type series of generator types I, II and III in a perspective view. They serve to elucidate the arrangement of the modules in different housings. The first type series I illustrated in FIG. 5a is a basic embodiment. In a rather smaller housing 12 there are arranged a module for the power supply 31 on one side, an inverter module 34 on the other side as individual modules 3 and in between the distribution module 20 as base module 2. A front panel of modular design with a display module 36 is arranged on the front side. A display control module 26 (not visible in FIGS. 5a-c, cf. FIGS. 4a-c) is arranged on the rear side of the display module 36. A rear-side module 27 for connection of external components is provided on a rear wall of the housing 12. A connection module 35 is plugged into the distribution module 20, and outputs the output energy generated by the inverter via an output connection 19.

FIG. 5b illustrates a medium-level type series II. The latter, by comparison with the type series I, has a different individual module for the inverter, namely one with a more powerful inverter 34′. Furthermore, a larger display module 36′ is provided, which is controlled by the display control module 26. All this necessitates a larger housing 12′. In addition, a further connection module 35 is plugged into the base module 2, and outputs output energy via a further output connection 19′.

FIG. 5c illustrates a higher-level type series III, which is similar to the type series II, but has a further output connection 19″. For this purpose, a further connection module 35 is provided, which is inserted into an extension unit 29 for the base module 2. Furthermore, an ultrasound module 38 arranged in the free space between the module for power supply 31′ and the inverter module 34′ is provided as optional module 14. It is connected via cable connections (not illustrated) to the communication network 4 and the electricity supply network 5. All this necessitates a large housing 12″. On the rear side thereof, for the purpose of outputting the ultrasound energy, an additional RF output connection 39 is provided, to which the ultrasound module 38 is connected for the purpose of outputting ultrasound energy.

As is evident from these examples, a multiplicity of different generator types and type series can thus be produced with few modules 2, 3 and small changes.

1. Modular generator system comprising at least two generator types of electrosurgical generators which differ with regard to their equipment and/or functions, the generator types each being designed for feed to at least one electrosurgical instrument and being composed of a plurality of generator components comprising a power supply and an inverter, fed by the power supply for generating an output energy which is passed to at least one output connection for connection of the electrosurgical instrument,
wherein
the generator types are each of modular design with a plurality of modules comprising
at least one base module, which is uniform for the different generator types and which comprises at least one communication unit for inter-module communication, and
at least one individual module, which is different depending on generator type, the individual module(s) being embodied at least as module for the power supply and/or module for the inverter and/or at least one module for the output connection,
the at least one base module being designed identically for the different generator types, and the different generator types differing with regard to their individual module equipment.

2. Modular generator system according to claim 1, wherein a display control module, an internal distribution module, and/or an external communication module for communication to the outside is provided as the base module.

3. Modular generator system according to claim 1, wherein a plurality of different individual modules are provided, which differ functionally and thus form kinds of individual modules.

4. Modular generator system according to claim 3, wherein different variants are provided at least within one kind of individual modules.

5. claim 1, wherein individual modules are provided for power supply; inverter; connections for electrosurgical instruments; and/or front panels including indicator and/or a display.

6. Modular generator system according to claim 1, wherein individual modules are individually exchangeable.

7. Modular generator system according to claim 1, wherein at least one optional module with additional functionality is provided, said optional module having an electricity supply and communication connections to other modules automatically established if it is in its mounted state.

8. Modular generator system according to claim 1, wherein the modular generator system comprises a plurality of different type series of generator types, for each of said type series dedicated individual modules are provided, which differ from the individual modules of another type series, wherein however the base module may be usable across type series.

9. Modular generator system according to claim 2, wherein at least one of the base modules has an electricity supply distribution unit for other modules and/or connectors for the at least one module for the output connection, wherein the connectors have connections for a communication network, an electricity supply network and/or optionally also the output energy line.

10. Modular generator system according to claim 1, wherein the base module and the individual modules each have a dedicated communication unit which in the mounted state form a communication network for inter-module communication with the communication units of the other modules, said communication network being self-configuring.

11. Modular generator system according to claim 1, wherein for the base module an optional extension unit is provided which carries at least high voltage for the output energy like the base module.

12. Modular generator system comprising at least two generator types of electrosurgical generators which differ with regard to their functions,
the generator types each being designed for supplying an electrosurgical instrument and being composed of a plurality of generator components comprising a power supply and an inverter, fed by the power supply for generating an output energy which is passed to at least one output connection for connection of the electrosurgical instrument,
wherein
the modular generator system comprises:
different inverter modules, one of these being selected and mounted for the respective generator type, and
different output connections, at least one of which being selected and mechanically accommodated in a front panel and electrically connected to the mounted inverter module,
each electrosurgical generator having a module detector which is configured for recognizing mounted and connected modules and determines therefrom a configuration of modules which are actually present.

13. Modular generator system according to claim 12, wherein the modules have a dedicated self-configuration unit designed to configure the respective module depending on the determined configuration of modules which are actually present.

14. Modular generator system according to claim 12, wherein the module detector is embodied as a central unit for the respective electrosurgical generator or in a decentralized manner on the respective modules.

15. Modular generator system according to claim 12, wherein a release unit is furthermore provided, which is designed, depending on the configuration of modules which are actually present, to determine enabled functions and to communicate corresponding release signals to the modules.

16. Modular generator system according to claim 15, wherein the release unit is embodied as a central unit for the respective electrosurgical generator or in a decentralized manner on the respective modules.

17. Modular generator system according to claim 12, wherein the modules are configured such that communication connections and an electricity supply of the modules are automatically established in the mounted state, wherein the communication connections are a communication network and additional signal connections provided for enable signals regarding activity of the modules.

18. Modular generator system according to claim 12, wherein the modules each have a dedicated processor unit with a dedicated control program as independent local operational control for the respective module, and they are connected to a generator-internal electricity supply network and communication network, wherein at least the inverter modules and/or optional ultrasound modules have an additional radio-frequency output connection.

19. Modular generator system according to claim 12, wherein a number of different power supply modules are furthermore provided, from which one is selected and mounted for the respective generator type.

20. Modular generator system according to claim 12, wherein a protection unit is provided which is designed to compare a configuration actually present with a unique, permanently stored target equipment feature for the configuration and, in case of any deviation, to output an error signal.

21. Modular generator system according to claim 20, wherein an upgrade unit is provided which is controllable externally via a secure communication connection and interacts with the protection unit and the self-configuration unit of the modules in order to provide the stored target equipment identifier feature with an authorized change and/or to release additional functions.
